(19) United States
(12) Patent Application Publication
(10) Pub. No.: US 2024/0293595 A1
(43) Pub. Date: Sep. 5, 2024

(54) MULTILAYER DEVICE FOR SUPPLYING NITRIC OXIDE

(71) Applicant: TO2M CORPORATION, Hsinchu (TW)

(72) Inventors: Garry TSAUR, Rowland Heights, CA (US); Ting-Hua WANG, Rowland Heights, CA (US)

(21) Appl. No.: 17/768,117

(22) PCT Filed: Oct. 9, 2019
(86) PCT No.: PCT/CN2019/110193

§ 371 Date: Apr. 11, 2022

(51) Int. Cl. A61L 15/44; A61K 33/00; A61L 15/22; B32B 15/08; B32B 15/18; B32B 15/20

(52) U.S. Cl. CPC A61L 15/44; A61K 33/00; A61L 15/225; B32B 15/08; B32B 15/18; B32B 15/20; B32B 2307/7242; B32B 2307/726; B32B 2307/73

(57) ABSTRACT

The present invention provides a multilayer device for supplying nitric oxide, which includes: an air-impermeable layer; a functional unit layer including a metal unit and a nitric acid inclusion unit, wherein dilute nitric acid can be released by destroying the nitric acid inclusion unit and reacts with the metal unit to generate nitric oxide gas, and the functional unit layer can further contain sulphites and can serve as a deoxidizing agent for absorbing oxygen and slowing down oxidation; a liquid absorption layer; and a hydrophobic air-permeable layer, a surface thereof being provided with a plurality of micropores allowing gas to pass therethrough, wherein the multilayer device may further include a skinfriendly layer arranged on the side of the hydrophobic air-permeable layer that is adjacent to a user. The multi-layer device can be used in hygiene products for everyday use.

### BACKGROUND OF THE INVENTION

### 1. Field of the Invention

The present invention relates to a multilayer device for supplying nitric oxide, which achieves the use of healthcare and disease improvement by supplying nitric oxide onto skin or into human body, and belongs to the field of life and healthcare.

### 2. Description of the Prior Art

In 1998, 3 American scientists, Robert Furchgott, Louis J. Ignarro and Ferid Murad, had the honour to win the Nobel Prize in Physiology or Medicine for their research results of nitric oxide (NO). Until now, the importance of NO in organisms has been widely confirmed.

Nitric oxide is an important physiological regulating substance in the immune system, nervous system and cardiovascular system. It can cause the effect of relaxing the vascular smooth muscle in living organisms, so as to ensure sufficient blood supply to the heart through vasodilation and vascular dilatation. It can regulate the blood supply to the myocardium, balance blood pressure and reduce vascular resistance, so as to achieve the effects of preventing angina pectoris, strengthening the lung function and controlling hypertension. Currently, it has been approved as a pulmonary vasodilator in some areas to treat neonatal diseases.

Nitric oxide is also a nerve conduction substance. It can transmit information among nerve cells and promote the communication among nerve cells, is closely related to the centers of brain learning, memory, sleep, coordination and the like. When applied to human body, nitric oxide can achieve the effects of promoting brain learning, improving memory, improving sleep quality, releasing stress and the like, and can affect the gastrointestinal digestive function, and even affect genital erection, relieve menstrual pain and prevent senile dementia.

Nitric oxide also has the effect of reducing insulin resistance, and improving insulin sensitivity to blood glucose, thereby accelerating the metabolism of blood glucose in the body to improve diabetes and complications thereof. It also has the ability of preventing cancer and inhibiting tumor growth. By regulating the cytotoxicity of macrophages to microorganisms and tumor cells, it can enhance the immune capacity of macrophages to achieve the purpose of preventing and defeating cancer. It is also helpful to diminish inflammation and relieve pain. In addition to mediating normal physiological functions, nitric oxide also involves in improving different pathophysiological states such as septic shock, hypertension, stroke and neurodegenerative diseases. It plays an important role in medical and health fields.

In order to apply the aforementioned effects of nitric oxide to the human body actually, it has always been an important issue in the art how to produce nitric oxide, and supply and apply it to biomedicine to achieve the aforementioned various effects.

Nitric oxide can be applied in biomedicine in various forms. Among them, an externally-applied form of nitric oxide has been proved to be able to help heal wounds, burns and scalds, treat sexual dysfunction in men and women, promote hair growth and act on sites needing vasodilation. For example, Taiwan patents TW 200841880 and TW 201417845 have revealed that nitrites, acid agents and reductants can act together to produce nitric oxide, and it can achieve the effect of treating burns and scalds by applying in an external patch form; US patent U.S. Pat. No. 9,801,902 also provides a topical nitric oxide patch, which generates nitric oxide through a nitrite reaction, which is then be used for treating wounds. However, because nitric oxide bears free radicals, its chemical properties are very active. After it contacts and reacts with oxygen in the air, corrosive and toxic nitrogen dioxide (NO₂) will be formed. Nitrogen dioxide will cause irritation when it comes into contact with human body. When inhaled, nitrogen dioxide will further lead to toxic reactions, and under exposure to nitrogen dioxide for a long time, adverse health effects will be further caused.

In order to prevent the nitric oxide gas released by the external patch from being converted into toxic nitrogen dioxide, it is also an important topic in the art how to prevent the oxygen between the patch to the skin from contacting and then reacting with nitric oxide.

### SUMMARY OF THE INVENTION

An objective of the present invention is to provide a device for supplying nitric oxide, which is applied in biomedicine in an externally-applied form and solves the aforementioned problems, so as to achieve various effects as previously mentioned in the disclosure.

In order to solve the aforementioned problems, the present inventor provides a multilayer device 1 for supplying nitric oxide, which includes:
an air-impermeable layer 2 located at an end away from a user; a liquid absorption layer 4 usable for absorbing excess liquid;
a functional unit layer 3 arranged between the air-impermeable layer 2 and the liquid absorption layer 4, which generates nitric oxide gas through a chemical reaction, the functional unit layer 3 including:
   a metal unit 32 and a nitric acid inclusion unit 31, wherein the metal unit 32 can react with nitric acid to generate nitric oxide;
   wherein the nitric acid inclusion unit 31 encapsulates nitric acid 313 therein, the nitric acid 313 can be released by destroying the nitric acid inclusion unit 31, and the nitric acid 313 is dilute nitric acid; and
a hydrophobic air-permeable layer 5, a surface thereof being provided with a plurality of micropores 51 allowing gas to pass therethrough, wherein the hydrophobic air-permeable layer 5 can be a single layer or a composite layer.

To achieve the objective of the present invention, the multilayer device 1 for supplying nitric oxide can further include a skinfriendly layer 6, which is hydrophilic and can be used for absorbing body fluid, and is arranged on a side of the hydrophobic air-permeable layer 5 that is adjacent to the user.

To achieve the objective of the present invention, the material of the air-impermeable layer 2 is polypropylene, a thin polyethylene plastic film, an aluminum film, or a composite film of a combination thereof.

To achieve the objective of the present invention, the functional unit layer 3 can further include a deoxidizer, a super absorbent polymer, a activator, or a catalyst.

To achieve the objective of the present invention, the deoxidizer includes an iron-based deoxidizer, a sulfite-based deoxidizer, or a deoxidizer of hydrogenation catalyst type.

To achieve the objective of the present invention, the deoxidizer is preferably the sulfite-based deoxidizer.

To achieve the objective of the present invention, the metal unit 32 is selected from a group consisting of sodium, magnesium, aluminum, zinc, iron, tin, lead, copper, silver and a combination thereof.

To achieve the objective of the present invention, the metal unit 32 can be powdery, granular, streaky or a thin film.

To achieve the objective of the present invention, the metal unit 32 can be located in the liquid absorption layer 4.

To achieve the objective of the present invention, there can be one or several nitric acid inclusion units 31.

To achieve the objective of the present invention, the nitric acid inclusion unit 31 can be spherical, elliptical, saccular or tubular.

To achieve the objective of the present invention, the nitric acid inclusion unit 31 is a metal composite film with high gas and water resistance, with an inner layer thereof being a polymer film 312 and an outer layer thereof being a metal film 311.

To achieve the objective of the present invention, the polymer film 312 is selected from a group consisting of polyethylene, polypropylene, polyethylene terephthalate (PET), a thin aluminized PET film, polysilazane-coated PET, a thin cast polypropylene film (CPP), a thin aluminized CPP film, aluminum oxide composite PET/CPP, copolyester plastic and a combination thereof.

To achieve the objective of the present invention, the material of the metal film 311 is selected from a group consisting of sodium, magnesium, aluminum, zinc, iron, tin, lead, copper, silver and a combination thereof.

To achieve the objective of the present invention, the material of the metal film 311 is preferably aluminum, iron or copper.

To achieve the objective of the present invention, the weight ratio of the nitric acid to water is 1:3-1:20, preferably 1:4-1:6, and most preferably 1:5.

To achieve the objective of the present invention, the skinfriendly layer 6 can be silicone, silica gel, a non-woven fabric, a cotton material or a plastic air-permeable film.

To achieve the objective of the present invention, the device can be used in a hygiene product for everyday use, and the hygiene product includes human body dressing, eye mask, mouth shad, face mask, facial mask, bra, breast patch, anti-galactorrhea patch, sanitary napkin (towel), paper diaper, sanitary pad, wound dressing, wound plast, dressing gauze and antidecubitus pad.

In addition to solving the aforementioned problems, the present invention also provides a way to generate nitric oxide through the reaction of dilute nitric acid and metal, and utilize it in an externally-applied device for supplying nitric oxide, which has the advantage of generating enough nitric oxide faster and more effectively than the reactants used in the patents of the prior art. The present invention further provides an inclusion unit for encapsulating dilute nitric acid, wherein the reaction is activated to generate nitric oxide as desired, and isolation with the liquid absorption layer makes it a safe externally-applied device for supplying nitric oxide.

Another advantage of the present invention lies in that, by dissolving nitrogen dioxide in the liquid and isolating with the liquid absorption layer or by a chemical reaction manner utilizing the deoxidizer, the problem that nitric oxide gas released from the externally-applied nitric oxide patch is easy to contact with the oxygen between the patch to the skin and then converted into irritating nitrogen dioxide gas, is solved.

The present invention further discloses the optimal reaction formula ratio and dosage required for obtaining the best healthcare effect of nitric oxide, and illustrates that the multilayer device for supplying nitric oxide can be applied in a hygiene product for everyday use, as well as the extended application of the present invention.

### BRIEF DESCRIPTION OF THE DRAWINGS

FIG. 1 is a perspective cross-sectional view of the multilayer device for supplying nitric oxide.

FIG. 2 is an exploded view of the multilayer device for supplying nitric oxide.

FIGS. 3A and 3B are cross-sectional views of the multilayer device for supplying nitric oxide.

FIG. 4 is a cross-sectional view of the nitric acid inclusion unit.

FIG. 5 is a schematic diagram of applying the multilayer device for supplying nitric oxide to human body dressing.

### DESCRIPTION OF MAIN NUMERICAL SYMBOLS

1 Multilayer device for supplying nitric oxide

3 Functional unit layer

31 Nitric acid inclusion unit

311 Metal film

312 Polymer film

313 Nitric acid

32 Metal unit

4 Liquid absorption layer

5 Hydrophobic air-permeable layer

51 Micropore

6 Skinfriendly layer

### DETAILED DESCRIPTION OF THE PREFERRED EMBODIMENT

Unless otherwise defined, all technical and scientific terms mentioned in the specification of the present invention have common meanings that can be generally understood by those of skills in the art.

The present invention is illustrated schematically by the following examples. It should be understood that the examples are only for explaining the technical ideas and characteristics of the present invention, are intended to enable those of skills in the art to understand the content of the present invention and implement it accordingly, and should not be constructed as limiting the patent scope of the present invention. That is, any equivalent changes or modifications made according to the spirit disclosed by the present invention should still be encompassed within the patent scope of the present invention.

### EXAMPLE 1

### Structure of Multilayer Device for Supplying Nitric Oxide

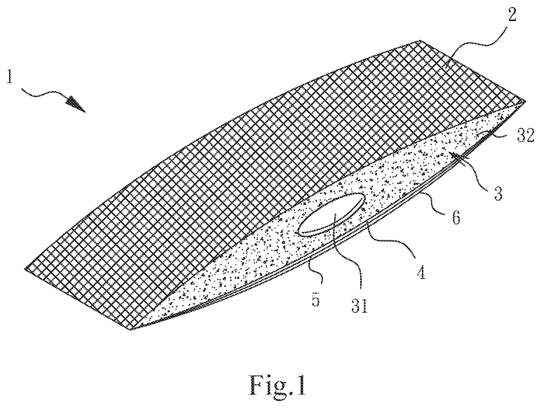

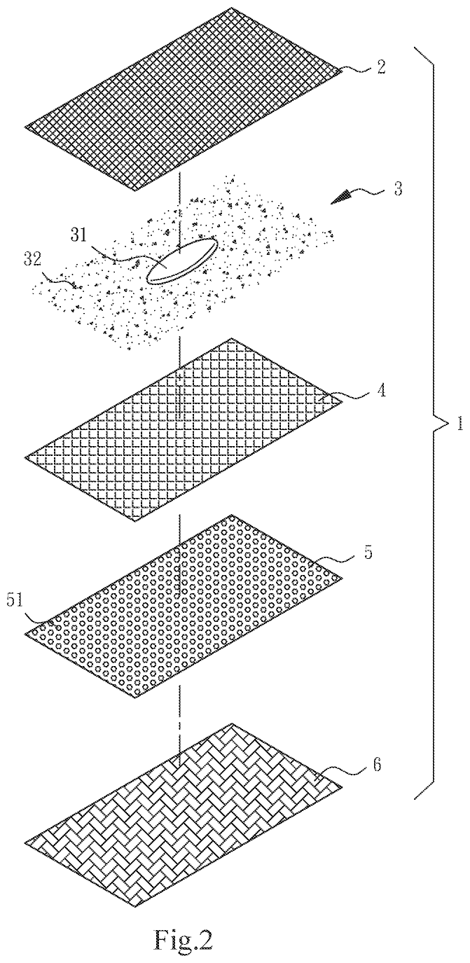

As shown in FIG. 1, the present inventor provides a multilayer device 1 for supplying nitric oxide, which includes:
an air-impermeable layer 2 located at the end away from a user, which is used for preventing the generated gas from releasing, wherein the material of the air-impermeable layer 2 is polypropylene, a thin polyethylene plastic film, an aluminum film, or a composite film of a combination thereof;
a liquid absorption layer 4 usable for absorbing excess liquid including reactants, products and water;
a functional unit layer 3 arranged between the air-impermeable layer 2 and the liquid absorption layer 4, which generates nitric oxide gas through a chemical reaction, the functional unit layer 3 including:
   a metal unit 32 and a nitric acid inclusion unit 31, wherein the metal unit 32 can react with nitric acid to generate nitric oxide;
   wherein the nitric acid inclusion unit 31 encapsulates nitric acid 313 therein, the nitric acid 313 can be released by destroying the nitric acid inclusion unit 31, and the nitric acid 313 is dilute nitric acid; and more precisely, the weight ratio of the nitric acid to water is 1:3-1:20, preferably 1:4-1:6, and most preferably 1:5; and
a hydrophobic air-permeable layer 5, a surface thereof being provided with a plurality of micropores 51 allowing gas to pass therethrough, wherein as shown in FIG. 2, the generated nitric oxide will be absorbed into the human body via the skin, nostrils, mouth, eyes and other contacted human sites through the plurality of micropores 51, and the hydrophobic air-permeable layer 5 can be a single layer or a composite layer.

According to the embodiments of the present invention, the multilayer device 1 for supplying nitric oxide can further include a skinfriendly layer 6, which is hydrophilic and can be used for absorbing body fluid, and is arranged on a side of the hydrophobic air-permeable layer 5 that is adjacent to the user.

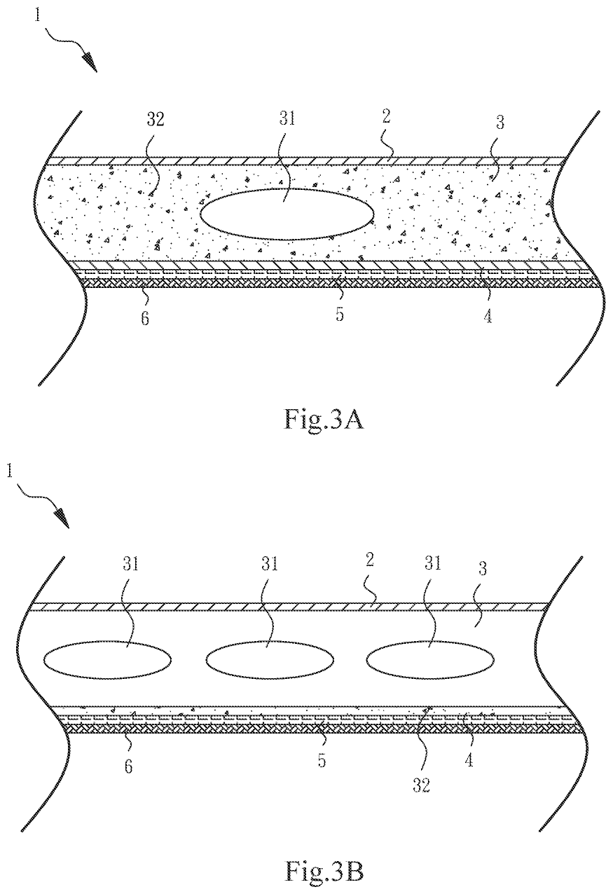

According to the embodiments of the present invention, the nitric acid inclusion unit 31 is used for encapsulating nitric acid 313. As shown in FIGS. 3A and 3B, there can be one or several nitric acid inclusion units 31, which can be spherical, elliptical, saccular or tubular, and stored in the functional unit layer 3.

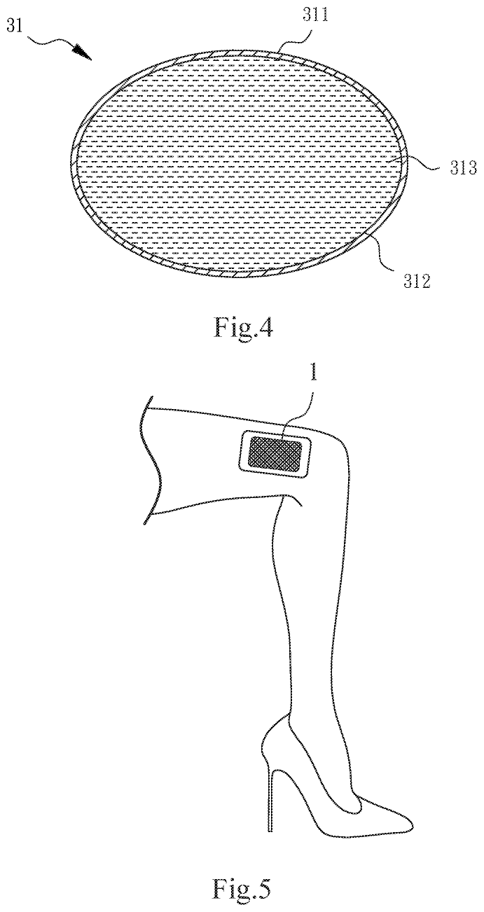

As shown in FIG. 4, the nitric acid inclusion unit 31 is a metal composite film with high gas and water resistance, with an inner layer thereof being a polymer film 312 and an outer layer thereof being a metal film 311; wherein, the material of the polymer film 312 is selected from a group consisting of polyethylene, polypropylene, polyethylene terephthalate (PET), a thin aluminized PET film, polysilazane-coated PET, a thin cast polypropylene film (CPP), a thin aluminized CPP film, aluminum oxide composite PET/CPP, copolyester plastic and a combination thereof; and wherein, the material of the metal film 311 is selected from a group consisting of sodium, magnesium, aluminum, zinc, iron, tin, lead, copper, silver and a combination thereof, and preferably aluminum, iron or copper.

### EXAMPLE 2

### Use Mode and Reaction of Multilayer Device for Supplying Nitric Oxide

According to the embodiments of the present invention, as desired, nitric acid can be released by destroying the nitric acid inclusion unit 31 in a manner including extruding, crushing or breaking. The released nitric acid can react to generate nitric oxide when it meets the metal unit 32, and the reaction is as follows:
3X+8HNO₃→3X(NO₃)₂+2NO↑+4H₂O
Y+4HNO₃→Y(NO₃)₃+NO↑+2H₂O
3Z+4HNO₃→3ZNO₃+NO↑+2H₂O
   wherein, X is magnesium, aluminum, zinc, iron, tin, lead or copper, Y is aluminum, iron or copper, and Z is sodium or silver; wherein, the nitric acid is dilute nitric acid, and more precisely, the weight ratio of the nitric acid to water is 1:3-1:20, preferably 1:4-1:6, and most preferably 1:5; wherein, the metal unit 32 can be powdery, granular, streaky or a thin film, which is selected from a group consisting of sodium, magnesium, aluminum, zinc, iron, tin, lead, copper, silver and a combination thereof; and wherein the metal unit 32 can also be located in the liquid absorption layer 4, as shown in FIG. 3B.

### EXAMPLE 3

### Prevention of Formation of Nitrogen Dioxide in the Externally-Applied Form

When the generated nitric oxide gas comes into contact with the oxygen between the patch to the skin, it is easy to be transformed into toxic and irritating nitrogen dioxide, and its reaction is as follows:
2NO+O₂→2NO₂

According to the embodiments of the present invention, the functional unit layer 3 can further includes a deoxidizer, a super absorbent polymer, a activator, or a catalyst, wherein the deoxidizer includes an iron-based deoxidizer, a sulfite-based deoxidizer, or a deoxidizer of hydrogenation catalyst type, and preferably the sulfite-based deoxidizer.

According to the embodiments of the present invention, a sulfite-based deoxidizer, such as sodium sulfite, which is a commonly used deoxidizer and can absorb oxygen and slow down the oxidation, is added into the functional unit layer 3. When it comes into contact with oxygen, it is easy to undergo the following reaction:

2Na₂SO₃+O₂→2Na₂SO₄

Through this reaction, the functional unit layer 3 can form an oxygen-free environment, so as to achieve the aforementioned objective of preventing the generated nitric oxide from being oxidized into toxic and corrosive nitrogen dioxide, and further preventing nitrogen dioxide from harming human body. This example can further generate nitric oxide intended to be produced in the present invention by reacting with dilute nitric acid, and the reaction is as follows:
3Na₂SO₃+2HNO₃→3Na₂SO₄+2NO↑+H₂O

The objective of supplying nitric oxide of the present invention can also be achieved by the reaction between dilute nitric acid and sodium sulfite.

According to the embodiments of the present invention, if nitrogen dioxide is still generated during use, the nitrogen dioxide will also react with water to form nitric oxide and nitric acid, and the reaction is as follows:
3NO₂+H₂O→2HNO₃+NO↑

The reaction can further prevent the release of toxic nitrogen dioxide and transform it into nitric oxide intended to be produced in the present invention. Through protection by the isolation with the liquid absorption layer 4 and the hydrophobic air-permeable layer 5, it can more effectively protect the safety of the user.

According to the embodiments of the present invention, the liquid absorption layer 4 is used for absorbing excess liquid including reactants, products and water, and the excess liquid cannot pass through the hydrophobic air-permeable layer 5, the hydrophobic air-permeable layer 5 further prevents a substance other than gas, such as nitrogen dioxide dissolved in the liquid, and the like, from contacting the skin and causing harm. However, the nitric oxide generated in the functional unit layer 3 can pass through the liquid absorption layer 4 and be released to the skinfriendly layer 6 through the micropores 51 in the hydrophobic air-permeable layer 5, so as to reach the skin of the user and supply nitric oxide to the skin in a targeted manner. The skinfriendly layer 6 can be silicone, silica gel, a non-woven fabric, a cotton material or a plastic air-permeable film.

According to the embodiments of the present invention, the multilayer device 1 for supplying nitric oxide can be used in a hygiene product for everyday use, and the hygiene product includes but is not limited to human body dressing (as shown in FIG. 5), eye mask, mouth shad, face mask, facial mask, bra, breast patch, anti-galactorrhea patch, sanitary napkin (towel), paper diaper, sanitary pad, wound dressing, wound plast, dressing gauze and antidecubitus pad; wherein, during storage, it is packed with an airtight material to further ensure that air will not contact with the multilayer device 1 for supplying nitric oxide; and when in use, the airtight package is torn off and it is applied to the required position.

In order to clarify a preferred dosage ratio, the nitric oxide generation efficiency is tested when each reactant in the range of 0.01-100 g. When the ratio of the aforementioned nitric oxide generation formula is dilute nitric acid: metal unit=1:100-100:1, nitric oxide can be generated by reaction; when the weight ratio is 1:10-10:1, it has preferred nitric oxide generation efficiency, and according to different metal units, it has the most preferred nitric oxide generation efficiency when the molar concentration ratio is 8:3, 4:1 or 4:3.

To sum up, the multilayer device 1 for supplying nitric oxide of the present invention, when applied in a hygiene product for everyday use, can cause the effect of relaxing the vascular smooth muscle by supplying nitric oxide. It can regulate blood supply, balance blood pressure and reduce blood vessel resistance to achieve the effects of preventing angina pectoris, strengthening lung function and controlling hypertension. It can also be used for improving neurological diseases, diabetes and complications thereof, side effects of radiotherapy, septic shock, stroke, constipation, ultraviolet injury, eye acor, eye discomfort, healing of burns and scalds, cancer prevention and anti-cancer efficacies, and even contribute to brain learning, memory, sleep, genital erection, relief of menstrual pain, enhancement of immunity and prevention of senile dementia, etc. The application of the multilayer device for supplying nitric oxide of the present invention can achieve excellent, extensive and diversified health care effects on body.

On the whole, the present invention can produce enough nitric oxide in a short time and supply it to the skin, nostrils, mouth, eyes or other sites of the human body that are in contact with it. It is convenient to carry around, prevents the formula from harming human body through multiple barriers, and can be used close to the skin. Moreover, it has simple and convenient generation of nitric oxide, and has optimized effects such as preventing nitric oxide from being derived into harmful by-products by means of isolation and deoxidation, etc.

For the diseases listed in the present invention, according to the embodiments of the present invention, the multilayer device 1 for supplying nitric oxide of the present invention can be reasonably applied to any disease caused by nitric oxide deficiency, such as cancer, side effects of chemotherapy, metabolic diseases, immune diseases, hypertension, diabetes, weight control, colitis, etc.

The present invention can also adjust the supply concentration of nitric oxide according to different needs of users. For example, nitric oxide can be used for adjusting the motility of sperm. Low concentration of nitric oxide can promote the activity of sperm, while high concentration of nitric oxide can inhibit the motility of sperm. By adjusting the concentration or use time of nitric oxide supplied by the present invention, it can have the effect of pregnancy assistance or contraception on the user.

In order to improve the nitric oxide generation effect, the functional unit layer 3 can optionally further include a super absorbent polymer, an activator or a catalyst, so as to meet different requirements and achieve the desired efficacy.

The aforementioned detailed description is directed to concrete description of the feasible examples of the present invention, but these examples are not intended to limit the patent scope of the present invention, and any equivalent implementation or change made without departing from the technical spirit of the present invention should be encompassed in the patent scope of the present invention.

The aforementioned multiple functions fully meet the statutory patentability of novelty and inventive step. Therefore, we apply for application according to the law, and urge your bureau to approve this patent application for invention to inspire the invention.

What is claimed is:

1. A multilayer device for supplying nitric oxide, comprising:
an air-impermeable layer located at an end away from a user;
a liquid absorption layer usable for absorbing excess liquid;
a functional unit layer arranged between the air-impermeable layer and the liquid absorption layer, which generates nitric oxide gas through a chemical reaction, the functional unit layer comprising:
a metal unit and a nitric acid inclusion unit, wherein the metal unit can react with nitric acid to generate nitric oxide;
wherein the nitric acid inclusion unit encapsulates nitric acid therein, the nitric acid can be released by destroying the nitric acid inclusion unit, and the nitric acid is dilute nitric acid; and
a hydrophobic air-permeable layer, a surface thereof being provided with a plurality of micropores allowing gas to pass therethrough, wherein the hydrophobic air-permeable layer can be a single layer or a composite layer.

2. The multilayer device for supplying nitric oxide according to claim 1, wherein the device can further comprise a skinfriendly layer, which is hydrophilic and can be used for absorbing body fluid, and is arranged on a side of the hydrophobic air-permeable layer that is adjacent to the user.

3. The multilayer device for supplying nitric oxide according to claim 2, wherein the skinfriendly layer can be silicone, silica gel, a non-woven fabric, a cotton material or a plastic air-permeable film.

4. The multilayer device for supplying nitric oxide according to claim 1, wherein the material of the air-impermeable layer is polypropylene, a thin polyethylene plastic film, an aluminum film, or a composite film of a combination thereof.

5. The multilayer device for supplying nitric oxide according to claim 1, wherein the functional unit layer can further comprise a deoxidizer, a super absorbent polymer, an activator, or a catalyst.

6. The multilayer device for supplying nitric oxide according to claim 5, wherein the deoxidizer comprises an iron-based deoxidizer, a sulfite-based deoxidizer, or a deoxidizer of hydrogenation catalyst type.

7. The multilayer device for supplying nitric oxide according to claim 6, wherein the deoxidizer is preferably the sulfite-based deoxidizer.

8. The multilayer device for supplying nitric oxide according to claim 1, wherein the metal unit is selected from a group consisting of sodium, magnesium, aluminum, zinc, iron, tin, lead, copper, silver and a combination thereof.

9. The multilayer device for supplying nitric oxide according to claim 1, wherein the metal unit can be powdery, granular, streaky or a thin film.

10. The multilayer device for supplying nitric oxide according to claim 1, wherein the metal unit can be located in the liquid absorption layer.

11. The multilayer device for supplying nitric oxide according to claim 1, wherein there can be one or several nitric acid inclusion units.

12. The multilayer device for supplying nitric oxide according to claim 1, wherein the nitric acid inclusion unit can be spherical, elliptical, saccular or tubular.

13. The multilayer device for supplying nitric oxide according to claim 1, wherein the nitric acid inclusion unit is a metal composite film with high gas and water resistance, with an inner layer thereof being a polymer film and an outer layer thereof being a metal film.

14. The multilayer device for supplying nitric oxide according to claim 13, wherein the polymer film of the nitric acid inclusion unit is selected from a group consisting of polyethylene, polypropylene, polyethylene terephthalate (PET), a thin aluminized PET film, polysilazane-coated PET, a thin cast polypropylene film (CPP), a thin aluminized CPP film, aluminum oxide composite PET/CPP, copolyester plastic and a combination thereof.

15. The multilayer device for supplying nitric oxide according to claim 13, wherein the material of the metal film is selected from a group consisting of sodium, magnesium, aluminum, zinc, iron, tin, lead, copper, silver and a combination thereof.

16. The multilayer device for supplying nitric oxide according to claim 15, wherein the material of the metal film is preferably aluminum, iron or copper.

17. The multilayer device for supplying nitric oxide according to claim 1, wherein the weight ratio of the nitric acid to water is 1:3-1:20.

18. The multilayer device for supplying nitric oxide according to claim 1, wherein the weight ratio of the nitric acid to water is preferably 1:4-1:6.

19. The multilayer device for supplying nitric oxide according to claim 1, wherein the weight ratio of the nitric acid to water is most preferably 1:5.

20. The multilayer device for supplying nitric oxide according to claim 1, wherein the multiplayer device can be used in a hygiene product for everyday use, and the hygiene product comprises human body dressing, eye mask, mouth shad, face mask, facial mask, bra, breast patch, anti-galactorrhea patch, sanitary napkin (towel), paper diaper, sanitary pad, wound dressing, wound plast, dressing gauze and antidecubitus pad.
